(19) United States
(12) Patent Application Publication
(10) Pub. No.: US 2026/0311874 A1
(43) Pub. Date: Oct. 8, 2026

(54) SYSTEMS AND METHODS FOR AUTOMATED HARMONIZATION OF BIOLOGICAL SAMPLE MEASUREMENTS USING OUT OF BAND BIOLOGICAL SAMPLE MEASUREMENTS

(71) Applicant: Alden Scientific, Inc., Cambridge, MA (US)

(72) Inventors: James A. Heywood, Newton, MA (US); Timothy E. Vaughan, Cambridge, MA (US); Kriston L. McGary, Flagstaff, AZ (US); Dean E. Cerrato, Boston, MA (US)

(73) Assignee: Alden Scientific, Inc., Cambridge, MA (US)

(21) Appl. No.: 19/441,276

(22) Filed: Jan. 6, 2026

Related U.S. Application Data
(60) Provisional application No. 63/783,690, filed on Apr. 4, 2025.

(51) Int. Cl. G16H 50/20

(52) U.S. Cl. CPC G16H 50/20

(57) ABSTRACT

Systems and methods for automated harmonization of biological sample measurements across multi-omics datasets align data from diverse measurement platforms, such as proteomics and genomics, by leveraging out-of-band health-related measurements (e.g., genomic data, clinical biomarkers) to correct biases and variability. A machine learning model identifies similar subjects across datasets based on health-related metrics, enabling dynamic scaling and adjustment of measurement-of-interest data. The harmonization process involves aligning statistical properties, such as mean and standard deviation, of target data with reference data to improve consistency and reliability. Applications include enhancing predictive model performance, integrating cross-platform datasets, and supporting clinical decision-making. The system can utilize genomic anchors, such as single nucleotide polymorphisms (SNPs), for robust corrections, ensuring accurate biomarker discovery and diagnostics.

### RELATED APPLICATIONS

This application claims priority to and the benefit of U.S. Provisional Application No. 63/783,690, filed Apr. 4, 2025, the contents of which is incorporated herein by reference in its entirety.

### FIELD OF TECHNOLOGY

Systems and methods herein related to technical fields of biological measurement and automated harmonization thereof using out of band neighboring samples, including machine learning-based harmonization, to compensate for relative quantitation, measurement bias, and other sources of error.

### BACKGROUND OF TECHNOLOGY

Omics is the collective characterization and quantification of sets of biological molecules and the investigation of how those molecules translate into the structure, function, and dynamics of an organism or group of organisms. Examples of fields of omics include genomics, proteomics, metabolomics, metagenomics, phenomics, transcriptomics, among others.

In omics, typically, advanced systems capable of measuring microscopic molecules are needed in order to measure and track the behaviors of the molecules in organisms. Such systems can be subject to various sources of error which can affect downstream analysis. Thus, error correction techniques can be used to increase the fidelity of measurements to actuality.

### SUMMARY

In some aspects, the techniques described herein relate to a method including: obtaining, by at least one processor, first measurement of interest data from at least one first multi-omics data source, the first measurement of interest data including a first plurality of measurement of interest measurements for a first plurality of measurement of interests of a first plurality of subjects in a first data set; obtaining, by at least one processor, first out-of-band health-related measurement data including a first plurality of health-related measurements for the first plurality of subjects in the first data set; obtaining, by at least one processor, second measurement of interest data from at least one second multi-omics data source, the second measurement of interest data including a second plurality of measurement of interest measurements for a second plurality of measurement of interests of a second plurality of subjects in a second data set; obtaining, by at least one processor, second out-of-band health-related measurement data including a second plurality of health-related measurements for the second plurality of subjects in the second data set; identifying, by the at least one processor, one or more particular first subjects of the first plurality of subjects, and one or more particular second subjects of the second plurality of subjects, utilizing at least one machine learning model based on: the first plurality of health-related measurement measurements of the first out-of-band health-related measurement data, and the second plurality of health-related measurement measurements of the second out-of-band health-related measurement data; comparing, by the at least one processor, the first measurement of interest data with the second measurement of interest data based on the first plurality of measurement of interest measurements and the second plurality of measurement of interest measurements associated with the one or more particular second subjects; and harmonizing, by the at least one processor, the one or more particular second subjects with the plurality of subjects by adjusting the first plurality of measurement of interest measurements.

In some embodiments, the term “in-band” refers to measurements of a same or common modality or omics relative the modality or omics of a target measurement.

In some embodiments, the term “out-of-band” refers to measurements of a different modality or omics than a target measurement.

In some embodiments, the term “panel” may refer to a set of proteins grouped according to function, such as functions created based on, e.g., predefined, configurable, derived, or other technique(s).

In some embodiments, the term “block” and/or “block data set” may refer to a set of proteomic measurements that are, or have been, obtained according to one or more common or shared experimental conditions, such as a common reagent batch, assay plate, sample processing protocol, or other condition or any combination thereof. A block may refer to a single experiment (e.g., a batch from an assay plate), a data set from the same reagent lot, a computational unit of measurement grouping, among other groupings based on experimental conditions.

In some embodiments, the term “in-block” refers to measurements obtained within a given block, and may refer to measurements of the same type (e.g., protein abundance, immunoassays, blood platelet counts, nucleotide measurements, etc.), or may be any measurement from within the block.

In some embodiments, the term “out-of-block” refers to measurements obtained from a block different from that of a given measurement, and may refer to measurements of the same type (e.g., protein abundance, immunoassays, blood platelet counts, nucleotide measurements, etc.), or may be any measurement from outside of the block of the given measurement.

In some embodiments, the term “assay” may refer to an investigative (e.g., analytic) procedure for qualitatively assessing or quantitatively measuring the presence, amount, or functional activity of a target entity, such as a protein measurement, block control value, or other analyte, measurand, or target, among others or any combination thereof.

In some embodiments, the term “block control” may refer to error correction, bias correction, or other pre-processing applied to a block and/or block data set.

In some embodiments, the term “sample” may refer to biological materials obtained from living or deceased subjects, and may include, without limitation, biofluids, tissue, cells, ribonucleic acid, deoxyribonucleic acid (DNA), protein lysate, cell-free DAN (cfDNA), circulating tumor DNA (ctDNA), among others or any combination thereof.

In some embodiments, “biofluid” may include, without limitation, blood, bile, bone Marrow Aspirate, breast milk, Cerebral spinal Fluid (CSF), plasma, saliva, serum, sputum, stool, swabs (oral, nasal, vaginal fluids), synovial fluid, urine, etc.

In some embodiments, “tissue” may include, without limitation, samples collected from a specific organ of the subject, such as, e.g., FFPE blocks, fixed tissue, fresh tissue, frozen tissue, slides, tissue microarrays (TMA), etc.

In some embodiments, the term “response variable,” “dependent variable,” “response measurement,” “dependent measurement,” “response protein,” “dependent protein,” or the like, may refer to a variable of interest to a particular study or analysis, such as the protein measurement value(s) for a particular protein of interest.

In some embodiments, the term “covariate” may refer to a variable that affects a response, or dependent, variable, but not of the variable of interest, such as a protein that influences, is influenced by, or is otherwise correlated with the protein measurement value(s) for a particular protein of interest.

In some embodiments, the term “bias” may refer to a systematic tendency in which the methods used to measure and/or gather data and generate statistics present an inaccurate, skewed or otherwise incorrect representation of actuality, such as a systematic offset to protein measurements.

### BRIEF DESCRIPTION OF THE DRAWINGS

Various embodiments of the present disclosure can be further explained with reference to the attached drawings, wherein like structures are referred to by like numerals throughout the several views. The drawings shown are not necessarily to scale, with emphasis instead generally being placed upon illustrating the principles of the present disclosure. Therefore, specific structural and functional details disclosed herein are not to be interpreted as limiting, but merely as a representative basis for teaching one skilled in the art to variously employ one or more illustrative embodiments.

FIG. 1A is a block diagram illustrating the system for automated harmonization of biological sample measurements using anchor biological sample measurements in accordance with one or more embodiments of the present disclosure.

FIG. 1B is a block diagram illustrating the system for automated harmonization of biological sample measurements using anchor biological sample measurements included reference sample selection in accordance with one or more embodiments of the present disclosure.

FIG. 1C is a block diagram illustrating the system for automated harmonization of biological sample measurements using anchor biological sample measurements including target sample selection in accordance with one or more embodiments of the present disclosure.

FIG. 2 is a schematic block diagram illustrating a system for automated harmonization of biological sample measurements using anchor biological sample measurements in accordance with one or more embodiments of the present disclosure.

FIG. 3 illustrates a schematic flow chart diagram of a method for harmonizing one or more anchor biological subjects in accordance with one or more embodiments of the present disclosure.

FIG. 4 is a block diagram illustrating the interaction between protein measurements, anchor proteins, and a harmonization module in accordance with one or more embodiments of the present disclosure.

FIG. 5 is a block diagram illustrating a system for scaling in biological molecule measurement systems in accordance with one or more embodiments of the present disclosure.

FIG. 6 depicts a graph showing predicted vs reported BMI in UKB cohort in accordance with one or more embodiments of the present disclosure.

FIG. 7 depicts a graph showing predicted vs reported BMI in Aurora cohort in accordance with one or more embodiments of the present disclosure.

FIG. 8 depicts a graph showing BMI vs. leptin for UKB and Aurora cohorts in accordance with one or more embodiments of the present disclosure.

FIG. 9 depicts a graph showing model performance as a function of Aurora leptin NPX shift. Performance is optimized over a broad range of shift values, from around 1.5-2.1 in accordance with one or more embodiments of the present disclosure.

FIG. 10 depicts a graph showing BMI vs. leptin for UKB and Aurora cohorts, where the Aurora leptin value has been shifted (by 1.85 NPX units) to maximize model performance in accordance with one or more embodiments of the present disclosure.

Referring to FIG. 11 depicts a graph showing predicted vs reported BMI in the Aurora cohort, with shifted Aurora leptin values in accordance with one or more embodiments of the present disclosure.

### DETAILED DESCRIPTION

Various detailed embodiments of the present disclosure, taken in conjunction with the accompanying FIGs., are disclosed herein; however, it is to be understood that the disclosed embodiments are merely illustrative. In addition, each of the examples given in connection with the various embodiments of the present disclosure is intended to be illustrative, and not restrictive.

Throughout the specification, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure.

In addition, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, the terms “and” and “or” may be used interchangeably to refer to a set of items in both the conjunctive and disjunctive in order to encompass the full description of combinations and alternatives of the items. By way of example, a set of items may be listed with the disjunctive “or”, or with the conjunction “and.” In either case, the set is to be interpreted as meaning each of the items singularly as alternatives, as well as any combination of the listed items.

In the field of biological measurement, particularly within the realm of omics and/or state biology, such as, genomics, metabolomics, metagenomics, phenomics, transcriptomics antibody and/or peptide arrays, RNA sequencing, etc., accurate data collection and analysis are important for understanding the complex interactions and functions of molecules, substances, or others within organisms. However, the measurement of large datasets collected by a single device may often be normalized and controlled internally, allowing measurements to be self-consistent. One such measurement may refer to relative quantification and/or absolute quantification of a biological data set. Another measurement may refer to abundance levels that can be fraught with challenges, including biases and errors introduced during the data collection process. With appropriate controls across experiments and/or samples, the relative quantification measurement may be consistent across the data set, but inaccuracies may exist when attempting to match these measurements to a different biological sample and/or data set. These inaccuracies can significantly impact the reliability of downstream analyses and the potential of harmonizing a plurality of multi-modal data sets (e.g., multi-omic data sets). For instance, when attempting to apply models trained on one cohort to another, discrepancies in measurement values can lead to inconsistent and unreliable results.

Biological datasets are collected in diverse contexts. Large datasets collected by a single research study are often normalized and controlled internally, allowing measurements to be self-consistent, e.g., a reported value of a measurement has the same biological meaning across samples. For example, one assay may measure protein concentration in a sample using an assay that provides relative rather than absolute quantification. With appropriate controls across experiments and/or samples, the relative quantification can be consistent across the dataset. Thus, if a protein is measured using an absolute quantitative assay, e.g., reporting mg/mL, in one sample, other samples with the same relative score can be inferred to have the same absolute concentration.

Large biological datasets using relative quantification are often available as reference datasets, but harmonizing a new dataset with the reference dataset when matching control samples presents a barrier to the use of the new dataset and reference dataset. Techniques herein provide a method for harmonization of multi-modal datasets (e.g., multi-omic datasets) such as where one or more measurements uses a modality providing absolute quantitation and one or more measurements only provides relative quantitation.

Inventors have appreciated that biological organisms can exist within tight constraints, and thus many biological measurements may be at least partially correlated. For example, the protein leptin controls many aspects of fat storage and utilization; thus, the probability distribution of plasma levels of leptin is highly constrained by knowing a person's body fat percentage.

In some embodiments, when multiple biological assays are used for coincident measures of the same biological sample (e.g., plasma levels of Leptin and albumin) or measure the same biological organism (e.g., body weight and plasma leptin level), it is possible to constrain the probability distribution of the relative measure by the absolute measure. Thus, in a multi-modal biological dataset, an assay with absolute quantitation serves as an out-of-band anchor constraining the probability of the relative measure. When a relative assay measures multiple biological entities simultaneously, we can also use in-band relationships to constrain the probability distribution of each relative measurement.

Techniques herein provide innovative approaches to data harmonization across different proteomics data sets, specifically addressing the challenge of measurement bias in protein data across multi-modal data sets, by leveraging the relationships of biological variance of the person, as opposed to variance of measurements. Such techniques can leverage cross-metric correlations determine an improvement to out-of-band relationships, thereby enabling the correction of biases introduced by scaling newly received data from external metrics to match and/or align with measurements associated with one or more particular proteins, where these external metrics may refer to one or more results of clinical measurements associated with the proteins within the relationships. The approach involves adjusting (i.e., scaling) the output of a calculation associated with the external metrics (e.g., clinical measurements) to match a standard deviation of one or more similar biological samples, where the one or more similar biological samples may refer to health-related metrics associated with differing modal data sets. By systematically modifying the relative measure of one or more selected similar samples, this method provides a more reliable framework for applying predictive models across diverse data sets, thus overcoming a significant impediment in the field of proteomics data analysis.

For example, when harmonizing a new multi-modal dataset to a reference multi-modal dataset, one can select biological samples that are most similar (“neighbors”) between the two datasets using “out-of-band” anchors and find the mean value of the relative measure in the reference dataset. The relative measure can then be centered (e.g., via adjustment of the mean) in the new dataset to match that of the mean of the selected similar samples from the reference dataset. Furthermore, the new data can be scaled (e.g., via adjustment of the standard deviation) to match the standard deviation of the neighboring samples from the reference set. Further, the performance of harmonization can be evaluated by measuring the improvement of how the “in-band” (e.g., the same modality and/or omic of the target measurement) relationships among the relative measures converge on the relative relationships in the reference dataset.

For example, in the field of proteomics, accurate measurement and analysis of protein data are important for various applications, including clinical diagnostics and biomedical research. However, a significant challenge arises when attempting to harmonize protein data across large biological data sets. This challenge is particularly evident when dealing with data from different proteomics platforms or measurement techniques, which may not be directly comparable due to biases and variations present in measurement processes and specifically using relative quantification techniques.

Traditional methods for harmonizing protein data often rely on direct numerical comparisons or simple statistical adjustments. These approaches can be inadequate, as they do not account for complex mixed-sample relationships, namely between proteins and phenotypic measurement values; or the specific biases introduced by different measurement platforms. As a result, the predictive models trained on one biological sample and/or cohort may not perform effectively when applied to another biological sample and/or cohort, limiting the utility of such models in broader applications.

The present techniques address these challenges by introducing a novel approach for harmonizing protein data across multi-modal data sets. This approach leverages cross-metric correlations to adjust for measurement biases in proteins, thereby improving the accuracy and reliability of protein measurements. By utilizing a protein error model that accounts for these correlations, the method enables more accurate predictions of protein measurement values, enhancing the performance of predictive models and/or harmonizing modules across the multi-modal datasets. This approach not only corrects for biases within the same block of proteins but also utilizes information from health-related data and measurements measured according to other modalities, providing a comprehensive solution to the problem of data harmonization in proteomics.

Harmonization techniques detailed herein can provide improvements on existing normalization techniques, such as batch correction in proteomics and calibration curves in metabolomics, among others. Such improvements include enabling cross-batch assays, cross-cohort integration with external reference data, among other improvements, or any combination thereof. For example, integrating data from across different proteomics platforms, such as Olink™, SomaLogic™, mass spectrometry, among others, each platform may produce different errors, different block controls and/or different measurement formats/representations, and thus may not be directly comparable. Integrating the harmonization techniques detailed herein into one or more proteomics platforms, and/or using the harmonization techniques detailed herein separately from the proteomics platform(s) may allow for more direct and accurate integration of data from output by each platform.

In one embodiment, the system for obtaining and processing in-band and out-of-band protein data can be implemented using a distributed computing architecture, where multiple processors are employed to handle large datasets from various proteomics data sources. This setup allows for parallel processing of in-band and out-of-band protein measurements, enhancing the efficiency and speed of data analysis. In another embodiment, the system may utilize a cloud-based platform to store and process the in-band and out-of-band protein data alongside one or more health-related metrics associated with a multi-modal data set, providing scalability and remote accessibility for users. The cloud-based system can dynamically allocate resources based on the computational demands of the protein error model scaling and prediction tasks. Additionally, the system could incorporate machine learning algorithms that are specifically tailored to handle the distinct characteristics of proteomics data, such as high dimensionality and noise. These algorithms can be designed to automatically adjust their parameters based on the anchor protein metrics and a plurality of external metrics associated with multi-modal data sets and associated correlations identified during the obtaining phase, thereby improving the accuracy of the predicted protein measurement values. Furthermore, the system might include a user interface that allows researchers to visualize the adjustment and scaling measurements and the impact of the adjustments on the protein data based on matching the plurality of external metrics, facilitating a deeper understanding of the harmonization process. This interface could be customizable, enabling users to select specific proteins or blocks of interest for detailed analysis.

In some embodiments, to evaluate the harmonization algorithm, a small biased subset of the reference dataset (e.g., only using samples from individuals with normal or lower BMI) can be randomly selected. The bias in the subset effectively shifts the mean level of some proteins (e.g., Leptin) in the subsample away from the reference sample. The protein levels in the subsample can be re-centered, effectively scrambling the relationships. The harmonization technique can then be applied using the out-of-band anchors (e.g., clinical labs, blood count, genetic information, phenotypic information, polygenic risk scores (PRS), SNP-based associations, epigenetic markers, among other biological and/or medical information), and the original means of the proteins are then compared to the means after harmonization where perfect harmonization would have meant an R-Squared of 1. With small subsets (e.g., 20 biased samples) and few neighbors (e.g., 3) the R-Squared can be found to be 0.3 or less, with larger subsets (e.g., 160) and more neighbors (e.g., 20) the R-Squared can be found to be above 0.8.

In some embodiments, genetic data, including single nucleotide polymorphisms (SNPs), polygenic risk scores (PRS), and whole-genome sequencing (WGS) data, may serve as out-of-band anchors. Because genetic variations remain constant over time, they provide a robust control for harmonizing proteomic measurements across datasets and platforms.

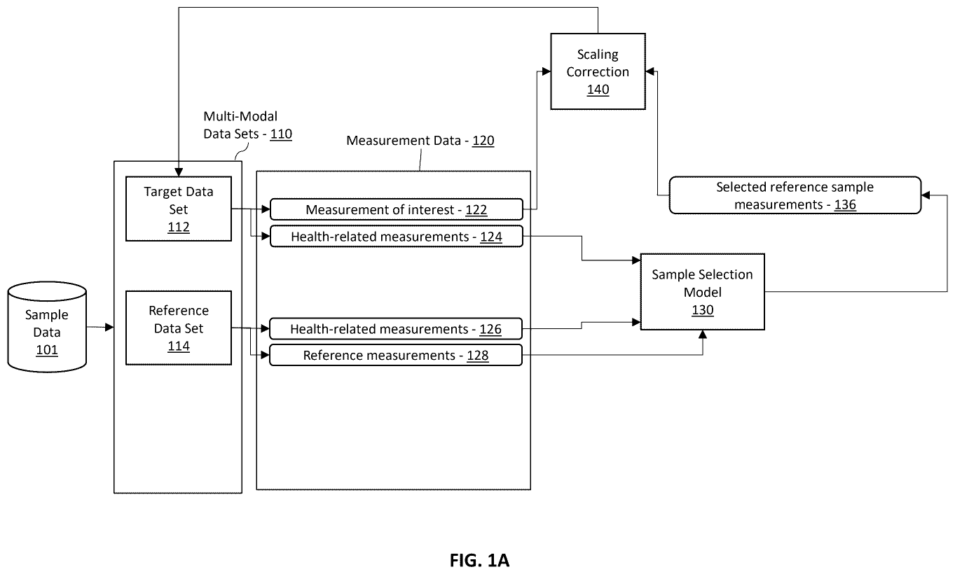

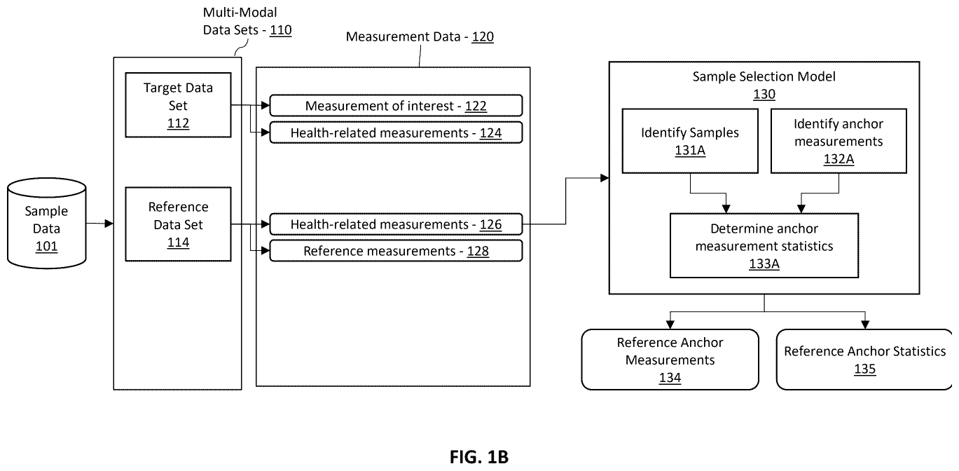

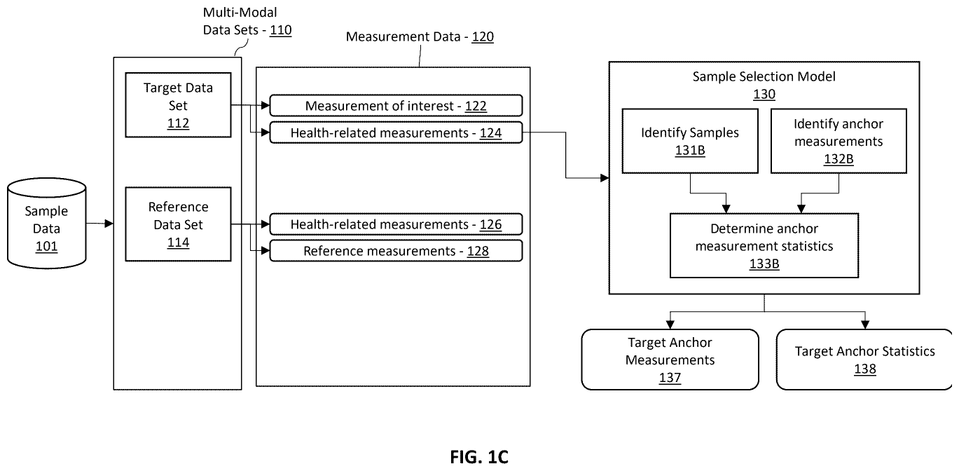

Referring now to FIG. 1, a block diagram is depicted that illustrates the use of a reference sample selection model 130 in automatic harmonization in protein measurement based on one or more external metrics, in accordance with one or more embodiments of the present disclosure.

One or more multi-or single-omics systems measure attributes of a material, compound, molecule or other substance of a biological being or any combination thereof. For example, proteomics platforms measure protein abundances. Such systems can produce data that is affected by attributes of the systems, including those that influence measurement error, bias, variability and other influences on the data. Moreover, the outputs of such systems may be represented in one or more of various ways, such as normalized, non-normalized, absolute, statistical and other representations or any combination thereof. As a result, one data set may produce measurements of the substance that are not directly comparable to the same measurements taken by another system.

Techniques herein leverage “anchor” measurements that can be used to scale one data set to match another such that the respective measurements are made more directly comparable (e.g., “harmonized”). For example, a measurement of interest can be scaled based on the relative differences in different “anchor” measurements between two data sets of interest. In some embodiments, the measurement of interest can be protein abundance, which may be represented differently between a target dataset and a reference dataset. Thus, anchor measurements can be used to determine a shift or scale in measurements between the target dataset and the reference dataset such that the protein abundances of the target dataset can be scaled to match the representation of the reference dataset.

In some embodiments, anchor measurements can include any measurement that is correlated to the variability of the measurement of interest and has a relatively consistent representation across systems. For example, weight, height, body-mass index, blood pressure, resting heart rate, age, sex, lab-based CRP, NMR metabolite data, imaging results, genetic information, phenotypic information, polygenic risk scores (PRS), SNP-based associations, epigenetic markers, among other biological and/or medical information among others are likely to be measured and represented similarly across datasets. Thus, to the extent such measurements are correlated to a measurement of interest, the anchor measurements can be used to align the measurement of interest across datasets by aligning probabilistic models, statistical models and/or other representations of the anchor measurements.

In some embodiments, in order to address misalignment between the target data set 112 and the reference data set 114, particularly with respect to the measurement of interest 122 relative to one or more reference measurements 128, scaling correction 140 is provided to a target data set 112. Sample data 101, e.g., collected by a multi-omics platform such as Olink™ or others, may be grouped in multi-modal data sets 110 having measurement data 120 from one or more samples. In certain embodiments, the multi-modal data sets 110 may relate to a measurement of one or more substances or factors for a particular sample, assay, panel, plate, reagent, or other block of measurement methodologies. In certain embodiments, the multi-modal data sets 110 may refer to a multi-modal data set that includes in-band biological measurements and out-of-band biological measurements.

In some embodiments, an anchor health-related measurement 126 in a reference data set 114 can be identified and used to determine, alone or in combination with other measurements, a scaling or adjustment to the measurement of interest 122 in the target data set 112 so as to re-align the measurement of interest 122 to match reference measurement 128 for the same substance or factor in the reference data set 114. To do so, correlated health-related measurements 124 that are correlated to the measurement of interest 122 can be identified and/or extracted from the target data set 112. Similarly, health-related measurements 126 correlated to the reference measurement(s) 128 can be extracted from the reference data set 114. The health-related measurements 124 and 128 may correspond to each other and be used to determine a degree of misalignment between the target data set 112 and the reference data set 114. Based on this degree of misalignment, a scaling correction 140 can be determined and applied to the measurement of interest 122 and/or to the reference measurement(s) 128 so as to bring the measurement of interest 122 and the reference measurement(s) 128 into alignment.

To do so, reference sample selection model 130 may be used to determine from the health-related measurements 124 and 128 the types of measurements that are correlated to the measurement of interest 122 and/or the reference measurement(s) 128. As a result, the reference sample selection model 130 may identify anchor, or calibrating, variables to calibrate the measurement of interest 122 and/or reference measurement(s) 128 so as to harmonize the representation of each.

For example, the reference data set 114 may include the UK Biobank (UKB), which has a large reference dataset with more than 50,000 participants that reports the value of various common clinical labs with absolute measurements in blood including the level of cholesterol, calcium, Vitamin D and clinical blood counts. The dataset also includes relative measures of 3,000 proteins in plasma. The inventors have a smaller dataset that they generated internally that measured the same absolute clinical labs and blood counts (out-of-band anchors) and the same relative measure of 3,000 proteins (in-band measures).

To harmonize the new data to the reference data, the most similar samples may be identified using the out-of-band anchors. The out-of-band anchors can be z-scored within the reference dataset to account for a range of biological variation observed across the reference population. The same scaling may be applied to the out-of-band anchors in the smaller dataset (from 20 to 160 samples). After scaling, distance measure between the samples in the reference and new datasets can be calculated (such as Euclidean, Jaccard, cosine, among others or any combination thereof). For each sample in the smaller dataset, multiple neighbors can be selected from the reference dataset (varying from 3-20 neighbors). The mean value for each protein may be calculated for the entire set of selected reference neighbors (e.g., average of 100 samples when the smaller dataset was 20 samples and 5 neighbors were selected for each sample) and the mean value of each protein in the smaller dataset can be shifted to match. After shifting the means for all proteins, the relationships between the protein levels can be compared to the relationship between the proteins in the reference dataset.

In some embodiments, the measurement of interest 122 can be identified and the predictive power of the correlated health-related measurement 124 can be leveraged, via a reference sample selection model 130, to determine a shift, calibration or other adjustment for the measurement of interest 122. However, out-of-band anchors for the measurement of interest 122 of the target data set 112 may be subject to the same block bias as the measurement of interest 122. Thus, correlated other data from reference data set 114 may be used. Herein, one reference data set 114 is depicted, though any number of data sets 110 may be present. By using the correlated data, e.g., health-related measurements 126 from the reference data set 114, the bias relative to the aggregate of all data sets 110 can be determined and used to calibrate target data set 112.

In some embodiments, the reference data set 114 may include any of one or more measurements associated with the protein of interest, such as, without limitation, e.g., biometric measurements, testing results, genomic measurements, phenotypic measurements, genetic information, phenotypic information, polygenic risk scores (PRS), SNP-based associations, epigenetic markers, among other biological and/or medical information among other health-related measurements or any combination thereof. For example, body mass index (BMI) may be correlated to a particular protein, and thus may be used as an out-of-band control for the protein measurements of the particular protein. Other out-of-band controls can include, e.g., body weight, height, blood pressure, heart rate, heart rate variability, cholesterol, blood glucose, among others or any combination thereof.

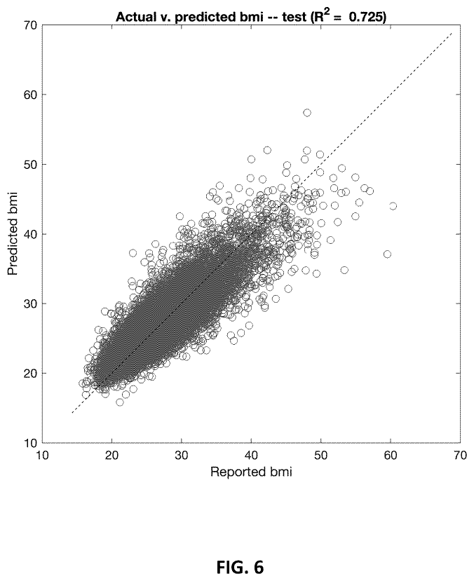

For example, when identifying out-of-band anchors for protein measurements, in some embodiments, one method to identify out-of-band controls can use an existing reference dataset to identify measures that are modeled by proteins effectively, such as by, a regression model of the candidate anchor using proteins has a high R² (see, for example, FIGS. 6-11, as detailed below). Proteins that are important features (e.g., contribute to information gain) in this model will be constrained by this anchor. A second approach can include identifying proteins associated with candidate anchors using prior knowledge (e.g., published papers).

Therefore, in some embodiments, the out-of-band health-related measurements 126 for the neighboring anchor measures from the other reference data set 114 can be used for their predictive power via correlation (i.e., similarity) to the measurement of interest. Thus, the out-of-band health-related measurements 126 can be input into a reference sample selection model 130 trained to determine selected reference sample measurements 136 of the health-related measurements 126 from the reference data set 114 that can be used to calibrate, via the scaling correction 140, for the measurement of interest 122.

In some embodiments, the reference sample selection model 130 of exemplary inventive computer-based systems and/or devices may be configured to utilize one or more exemplary AI/machine learning techniques chosen from, but not limited to, Bayesian networks, reinforcement learning, autoencoders, linear regression models, non-linear regression models, decision trees, boosting, support-vector machines, neural networks, nearest neighbor algorithms, Naive Bayes, bagging, random forests, and the like. In some embodiments and, optionally, in combination of any embodiment described above or below, an exemplary neural network technique may be one of, without limitation, feedforward neural network, radial basis function network, recurrent neural network, convolutional network (e.g., U-net) or other suitable network. In some embodiments and, optionally, in combination of any embodiment described above or below, an exemplary implementation of Neural Network may be executed as follows:
i) define Neural Network architecture/model,
ii) transfer the input data to the exemplary neural network model,
iii) train the exemplary model incrementally,
iv) determine the accuracy for a specific number of timesteps,
v) apply the exemplary trained model to process the newly-received input data,
vi) optionally and in parallel, continue to train the exemplary trained model with a predetermined periodicity.

In some embodiments and, optionally, in combination of any embodiment described above or below, the exemplary trained neural network model may specify a neural network by at least a neural network topology, a series of activation functions, and connection weights. For example, the topology of a neural network may include a configuration of nodes of the neural network and connections between such nodes. In some embodiments and, optionally, in combination of any embodiment described above or below, the exemplary trained neural network model may also be specified to include other parameters, including but not limited to, bias values/functions and/or aggregation functions. For example, an activation function of a node may be a step function, sine function, continuous or piecewise linear function, sigmoid function, hyperbolic tangent function, or other type of mathematical function that represents a threshold at which the node is activated. In some embodiments and, optionally, in combination of any embodiment described above or below, the exemplary aggregation function may be a mathematical function that combines (e.g., sum, product, etc.) input signals to the node. In some embodiments and, optionally, in combination of any embodiment described above or below, an output of the exemplary aggregation function may be used as input to the exemplary activation function. In some embodiments and, optionally, in combination of any embodiment described above or below, the bias may be a constant value or function that may be used by the aggregation function and/or the activation function to make the node more or less likely to be activated.

For example, in some embodiments, the reference sample selection model 130 may be specifically tailored to a protein of interest via training on the out-of-block health-related data measurements 126 determined to be associated with the protein of interest, e.g., from a training dataset such as the United Kingdom Biobank (UKB). Additionally, or alternatively, a multi-moda 1 data set may be able to train a plurality of predictive models for multiple proteins of interest and can be reused to make predictions and/or verify past predictions for multiple different proteins. In certain embodiments, the multi-modal data set may be utilized to scale the measurements of the out-of-band proteins to a plurality of proteins similar to the protein of interest.

In some embodiments, scaling correction 140 may be performed based on the constraint of a probability distribution of out-of-band and/or external metrics that are similar to the selected reference sample measurements 136 and the measurement of interest 122. However, in some embodiments, other errors and measurements may be present in a given measurement of interest 122 for the measurement of interest, e.g., due to additional measurement error, anomalies, artifacts, noise and other sources of error. In certain embodiments, the other measurements may refer to relative quantification of the measurement of interest. Thus, the protein measurement(s) 122 and the predicted protein measurement(s) 136 for the measurement of interest may be used with the protein measurement(s) 122 and the predicted protein measurement(s) 136 for additional measurement(s) of interest from any protein within the multi-modal data set. By obtaining selected reference sample measurements 136 for one or more measurement(s) of interest in the target data set 112, a calculated measurement between each selected reference sample measurements 136 and each actual measurement of interest 122 can be statistically aggregated to compare and verify the measurement of interest 122. For example, the difference between the selected reference sample measurements 136 and the actual measurement of interest 122, for example of each protein, from the target data set 112 can be averaged to generate an average bias for measurement of interest 122 in the target data set 112. Other forms of statistical aggregation can be used, such as weighted average, etc.

In some embodiments, upon determining the measurements, the scaling correction 140 can produce an offset to offset the bias in measurement values. As a result, the offset can be applied to the target data set 112 to offset bias in each measurement of interest 122. As a result, the correlation of other health-related measurements 124 and 126 can be leveraged to determine a likely misalignment in measurement between the measurement of interest 122 and the reference measurements 128 such that the measurement of interest 122 may be recalibrated, realigned, recentered, or otherwise scaled to harmonize with the reference measurements 128. For example, in some embodiments, protein measurements can be better harmonized with other data sets associated with the external metrics related to health-related measurements, be more accurately and reliably used for proteomics applications, such as new drug discovery, biomarker analysis, diagnostics, among other applications or any combination thereof. For example, the protein measurements can be leveraged to determine a likely condition, risk or prognosis associated with a patient from which the sample was collected. Such condition, risk or prognosis can be more accurate with the use of unbiased protein measurements. Similarly, harmonization of other measurements of interest 122 with a reference data set can be used to determine or recommend health-related studies, develop new drugs and treatments, develop diagnostics, assess efficacy of existing or new treatments and diagnostics, among other recommendations and determinations or any combination thereof.

In some embodiments, the scaling correction 140 can utilize one or more AI/ML techniques, such as those detailed above. In some embodiments, the AI/ML model used for scaling correction 140 can be the same as for the reference sample selection model 130 such that the model performs both sample selection and scaling. In some embodiments, the AI/ML model used for scaling correction 140 can be distinct from the AI/ML model used for the reference sample selection model 130 for a two step process. In some embodiments, the scaling correction 140 AI/ML model may be trained to ingest the correlated health-related measurement 124, such as genomic data, phenotypic data, metabolomic data, clinical data, etc. to predict a scaling correction to the measurement of interest 122.

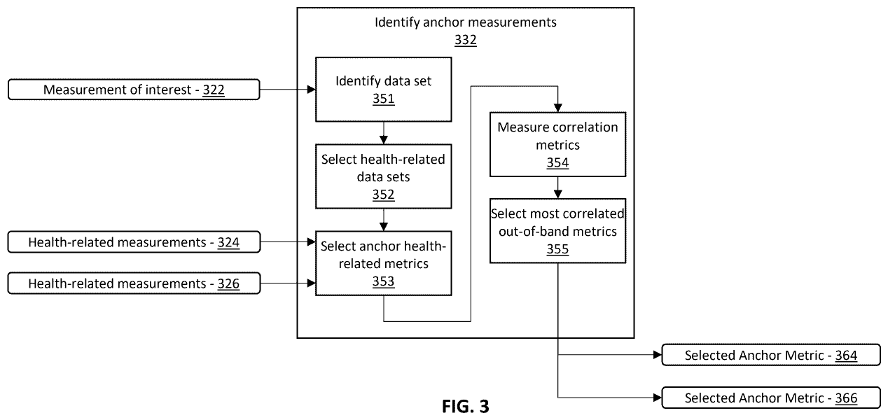

In some embodiments, the sample selection model 130 may be used to determine samples and anchor measurements from the reference data set 114 associated with the measurement of interest 122. As detailed above, the associated samples may be determined according to the anchor measurements correlated to the measurement of interest 122. In particular, health-related measurements different from the measurement interest 122 that behave in a way that is correlated to the behavior of the measurement of interest 122 may be used to infer probabilistic or statistical constraints on the measurement of interest 122. Thus, where such anchor measurements are identified (e.g., according to the process of FIG. 3, detailed below), the samples that are most correlated between the target data set 112 and the reference data set 114 may be identified as the samples having health-related measurements 124 and 126 most likely to be predictive of the degree of alignment between the target data set 112 and the reference data set 114.

Referring now to FIG. 1B, a block diagram is depicted illustrating the selection and analysis, using the sample selection model 130, of a reference sample and associated anchor measurements of the system for automated harmonization of biological sample measurements in accordance with one or more embodiments of the present disclosure.

In some embodiments, the sample selection model 130 may obtain the health-related measurements 126 of the reference data set 114, including sample data associated with each sample in the reference data set 114. Accordingly, the sample selection model 130 can identify each health-related measurement 126 according to its associated sample by identifying samples 131A. In some embodiments, identifying samples 131A may include identifying the data associated with samples having particular attributes (e.g., date, patient age, weight, sex, BMI, etc., among other attributes of the data or any combination thereof), identifying samples having particular identifiers, identifying samples based on quality of data or sample collection/processing method, identifying samples based on measurement systems/equipment, among other selection methodology or any combination thereof.

In some embodiments, the sample selection model 130 may identify anchor measurements 132A associated with the samples. In some embodiments, the anchor measurements 132A may include a subset of the measurements of each sample and may be selected according to the predictive power or degree of correlation to the measurement of interest 122. For example, the anchor measurements may be identified based on a correlation analysis in a training data set or in the reference data set 114, or other data set having validated measurement data, such as in the correlation analysis detailed with reference to FIG. 3 below. In another example, the anchor measurements may be identified based on predefined relationships, e.g., obtain from scientific study or other source. In some embodiments, the anchor measurements may be identified by a combination of correlation analysis, scientific study, or any other technique or any combination thereof.

In some embodiments, the sample selection model 130 may use the reference anchor measurements 134 for the identified samples to determine anchor measurement statistics 133A for the sample. In some embodiments, the anchor measurement statistics 135 can include, e.g., a mean, standard deviation, median, range, z-score, or other statistical analytic or any combination thereof. For example, the sample selection model 130 may determine anchor measurement statistics representative of the anchor measurements across the identified samples, such as mean and standard deviation. Alternatively, or in addition, the anchor measurement(s) of each sample can be analyzed relative to the population of anchor measurements, e.g., of the identified samples or of the data set as a whole or both, such as a z-score or other statistical scoring. Thus, the sample selection model 130 may output data indicative of the reference anchor measurements 134, including reference anchor measurement statistics 135.

Referring now to FIG. 1C, a block diagram is depicted illustrating the selection and analysis, using the sample selection model 130, of a target sample and associated anchor measurements of the system for automated harmonization of biological sample measurements in accordance with one or more embodiments of the present disclosure.

In some embodiments, similar to FIG. 1B, the sample selection model 130 may obtain the health-related measurements 126 of the target data set 112, including sample data associated with each sample in the target data set 112. Accordingly, the sample selection model 130 can identify each health-related measurement 126 according to its associated sample by identifying samples 131B. In some embodiments, identifying samples 131B may include identifying the data associated with samples having particular attributes (e.g., date, patient age, weight, sex, BMI, etc., among other attributes of the data or any combination thereof), identifying samples having particular identifiers, identifying samples based on quality of data or sample collection/processing method, identifying samples based on measurement systems/equipment, among other selection methodology or any combination thereof.

In some embodiments, the sample selection model 130 may identify anchor measurements 132B associated with the samples. In some embodiments, the anchor measurements 132B may include a subset of the measurements of each sample and may be selected according to the predictive power or degree of correlation to the measurement of interest 122. For example, the anchor measurements may be identified based on a correlation analysis in a training data set or in the target data set 112, or other data set having validated measurement data, such as in the correlation analysis detailed with reference to FIG. 3 below. In another example, the anchor measurements may be identified based on predefined relationships, e.g., obtain from scientific study or other source. In some embodiments, the anchor measurements may be identified by a combination of correlation analysis, scientific study, or any other technique or any combination thereof.

In some embodiments, the sample selection model 130 may use the target anchor measurements 137 for the identified samples to determine anchor measurement statistics 133B for the sample. In some embodiments, the target anchor statistics 138 can include, e.g., a mean, standard deviation, median, range, z-score, or other statistical analytic or any combination thereof. For example, the sample selection model 130 may determine anchor measurement statistics representative of the anchor measurements across the identified samples, such as mean and standard deviation. Alternatively, or in addition, the anchor measurement(s) of each sample can be analyzed relative to the population of anchor measurements, e.g., of the identified samples or of the data set as a whole or both, such as a z-score or other statistical scoring. Thus, the sample selection model 130 may output data indicative of the target anchor measurements 137, including target anchor statistics 138.

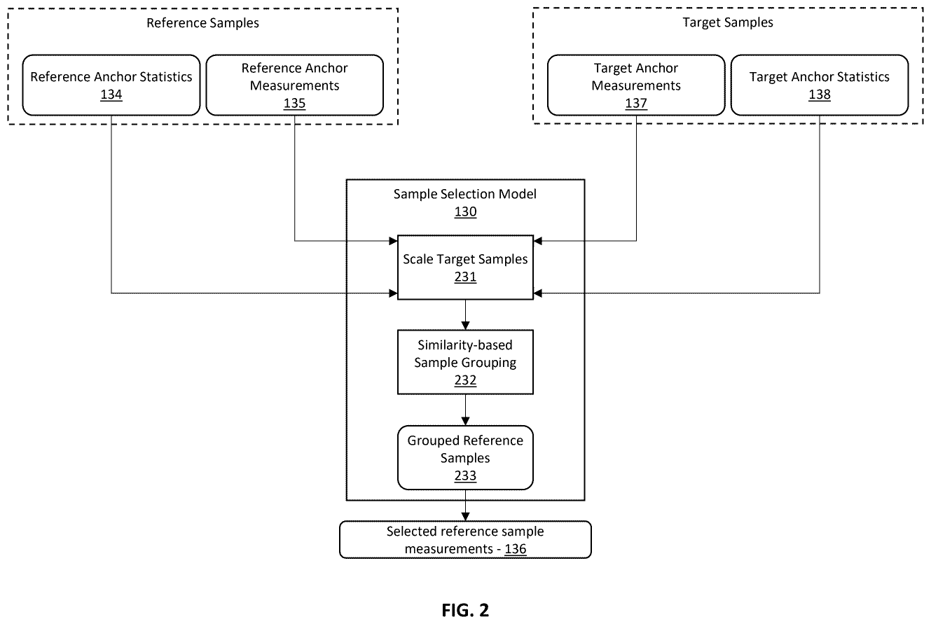

Referring now to FIG. 2, a block diagram is depicted that illustrates automated harmonization of biological sample measurements using health-related external metrics, in accordance with one or more embodiments of the present disclosure.

In some embodiments, the sample selection model 130 may use the reference anchor statistics 134, the reference anchor measurements 135, the target anchor measurements 137 and the target anchor statistics 138 to determine the most predictive reference samples to use as the selected reference sample measurements 136. In some embodiments, the selection may be based on a similarity of one or more reference samples to the target samples based on the similarity of the respective anchor statistics.

In some embodiments, the anchor measurements used for sample selection can be one or more different measurements, including multiple measurements of the sample type, multiple types of measurements, or both. For example, the anchor measurements can include a presence or absence of a particular gene, a presence or absence of multiple genes, a presence or absence of multiple genes as well as one or more additional clinical measurements. Indeed, utilizing genomics may facilitate particularly effective anchoring of the measurement of interest due the relatively large number of genes that can be measurements and may be correlated to the measurement of interest. For example, a discussion of proteomic disease signatures, prediction modelling and their relationship to genetics and health can be found in Sun BB, et al. Plasma proteomic associations with genetics and health in the UK Biobank. Nature. 2023 October; 622(7982): 329-338. doi: 10.1038/s 41586-023-06592-6. Epub 2023 Oct. 4. PMID: 37794186; PMCID: PMC 10567551., which is incorporated herein by reference in its entirety for all purposes. Therefore, by formulating an anchor (e.g., in vector form, or other format) from tens, hundreds, thousands, tens of thousands, hundreds of thousands, millions or more gene measurements correlated to the measurement of interest 122, the scaling of the measurement of interest 122 can be accurately and reliably anchored to the reference data set 114. Unlike existing harmonization methods that rely solely on clinical biomarkers, embodiments herein uniquely integrates genetic data as a stable reference. Genetic variants, which do not change over time, provide a more reliable out-of-band correction for proteomic variability, particularly in multi-cohort and cross-platform analyses. This approach enables superior alignment of protein data across diverse study populations, ensuring consistency in biomarker discovery and clinical decision support.

In some embodiments, to determine similarity, the sample selection model 130 may scale the target samples 231 to normalize the target anchor measurements 137 to the reference anchor measurements 135. For example, the reference anchor measurements 135 may be z-scored based on the reference anchor statistics 134, e.g., to z-score the reference anchor measurements 135 within the identified reference samples, within the reference data set 114, or both. Using the z-scores of the reference anchor measurements, the target anchor measurements 137 may be scaled correspondingly. Alternatively, or in addition, the z-score may be used to scale the target anchor measurements 137 within the target data set 112.

In some embodiments, a similarity measure can be applied to the reference samples and the target samples based on the scaled reference anchor measurements 135 and the scaled target anchor measurements 137, respectively. The similarity measure may be used for similarity-based sample grouping 232 to group the most similar reference samples with the target samples.

In some embodiments, the similarity measure may be used to group the reference samples to the target samples. For example, the similarity algorithm may be employed to determine similarity, which reference samples within a threshold distance of the target samples may be included in the group of reference samples. In some embodiments, the similarity measure be, e.g., Jaccard similarity, Jaro-Winkler similarity, Cosine similarity, Euclidean similarity, Overlap similarity, Pearson similarity, Approximate Nearest Neighbors, K-Nearest Neighbors, among other similarity measure. In some embodiments, a set of similar reference samples may be identified for each target sample using the similarity measure. In some embodiments, the grouping may be performed using, e.g., machine learning and/or clustering, such as using neural networks, autoencoders, support vector machines, among other AI/ML techniques, including those detailed above, or any other classification algorithm or any combination thereof.

In some embodiments, as a result of the similarity-based grouping 232, grouped reference samples 233 may be identified. The grouped reference samples 233 may include the reference samples that have similar characteristics to the target samples based on the anchor measurements. Because the anchor measurements are correlated to the measurement of interest, the variation of the anchor measurements can be used as a proxy for assessing the likelihood that a given reference sample would exhibit similar behavior in the measurement of interest to a particular target sample. Thus, by performing the similarity-based grouping 232 and output the reference samples having similar anchor measurements to the target sample, the sample selection model 130 may identify the reference samples most likely to have similarity in the measurement of interest.

As a result, the sample selection model 130 may produce the selected reference sample measurements 136 for use in scaling the measurement of interest 122.

Referring now to FIG. 3, another block diagram is depicted that illustrates operational steps for harmonizing one or more health-related external metrics, in accordance with one or more embodiments of the present disclosure.

In some embodiments, the measurement(s) of interest 322 may represent the actual measured values of a measurement of interest within a specific block, which may refer to the absolute quantification values. In certain embodiments, the protein measurements 322 may serve as the baseline data from which centering and/or scaling associated with anchor proteins are assessed. In certain embodiments, the protein measurements 322 may assist in identifying systematic biases and automatic harmonization between out-of-band proteins and in-band proteins. In some embodiments, the automatic harmonization via the protein measurements 322 may provide data for training models that predict expected protein values when such biases are not present.

In some embodiments, the out-of-band health-related measurements 324 and 326 may include measurements taken from proteins that are not within the same block as the protein of interest, thus providing relative quantification values capable of comparison and modification related to the absolute quantification values of the protein measurements 322. By analyzing these out-of-band health-related measurements 324 and 326, the system can identify proteins that have a predictive relationship with the protein of interest. These relationships are leveraged to adjust the protein measurements, thereby reducing the impact of block-specific biases. The selection of appropriate anchor proteins is important, as this choice influences the accuracy of the predicted protein measurements.

In some embodiments, the harmonization determination 350 component may refer to a mechanism that identifies and quantifies the relationships between the protein of interest and potential anchor proteins. In some embodiments, the component may involve several sub-processes, including the identification of the data set containing the protein of interest (351), the selection of health-related data sets for potential health-related external metrics (352), and the selection of health-related external metrics (353). The harmonization determination process measures measurement values (354) to assess the strength of the relationships between the protein of interest and candidate health-related external metrics. The external metrics with the lowest measured standard deviation value from the measurements associated with the protein of interest 351 are then selected (355) for use in the prediction model. This process can ensure that the prediction model is based on the most relevant and informative data.

In some embodiments, the selected health-related external metrics 364 and 366 components may represent the results of health-related external metrics identified as having a low standard deviation of measurement values with the protein of interest. These external metrics may be selected based on their standard deviation values and/or measurement values, which indicate the strength and direction of their relationship with the protein of interest. The health-related external metrics may be used as inputs to the protein measurement prediction model, providing the data needed to predict the expected protein measurement values (i.e., relative quantification values). In some embodiments, the selection of the health-related external metrics can influence the effectiveness of the bias correction and the accuracy of the predicted protein measurements.

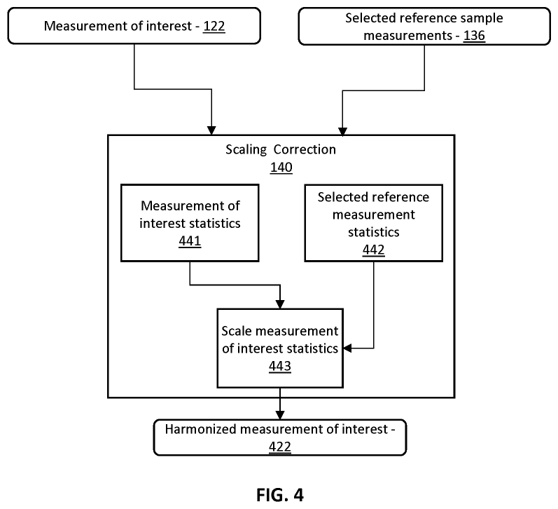

Referring to FIG. 4, a block diagram is depicted that illustrates the interaction between measurements of interest, anchor health-related measurements, and a prediction model with a harmonizer in accordance with one or more embodiments of the present disclosure.

In some embodiments, the scaling correction 140 may obtain the measurement of interest 122 of the target data set 112 and the selected reference sample measurements 136 of the reference data set 114, and in particular the reference sample measurement of the same type as the measurement of interest 122.

To harmonize the measurement of interest 122 to the reference data set 114, the scaling correction 140 may determine the statistics for each of the measurement of interest 122 and the selected reference sample measurements 136 across the respective samples. Indeed, the scaling correction 140 may determine measurement of interest statistics 441 across the target samples, including, e.g., mean, median, standard deviation, among others or any combination thereof. Similarly, the scaling correction 140 may determine selected reference measurement statistics 442 across the selected reference samples, including, e.g., mean, median, standard deviation, among others or any combination thereof.

In some embodiments, because the target samples and the references are identified as similar, then, in aggregate, the target samples and the references may be inferred to have similar behavior of the measurement of interest. Thus, while absolute values may be different, the statistical distribution may be similar. Accordingly, the scaling correction 140 may use this assumption to scale the mean and/or standard deviation of the measurement of interest 122 of the target data set 112 to match that of the selected reference sample measurements 136. For example, a multiplier, weight, coefficient, added constant, among other scalings may be applied to the mean, median and/or standard deviation of the measurement of interest statistics 441 so as to harmonize the measurement of interest statistics 441 with the distribution of the selected reference measurement statistics 442

In some embodiments, upon scaling the measurement of interest statistics 443, the measurement of interest 122 can be harmonized based on the scaled measurement of interest statistics, thus harmonizing the target data set 112.

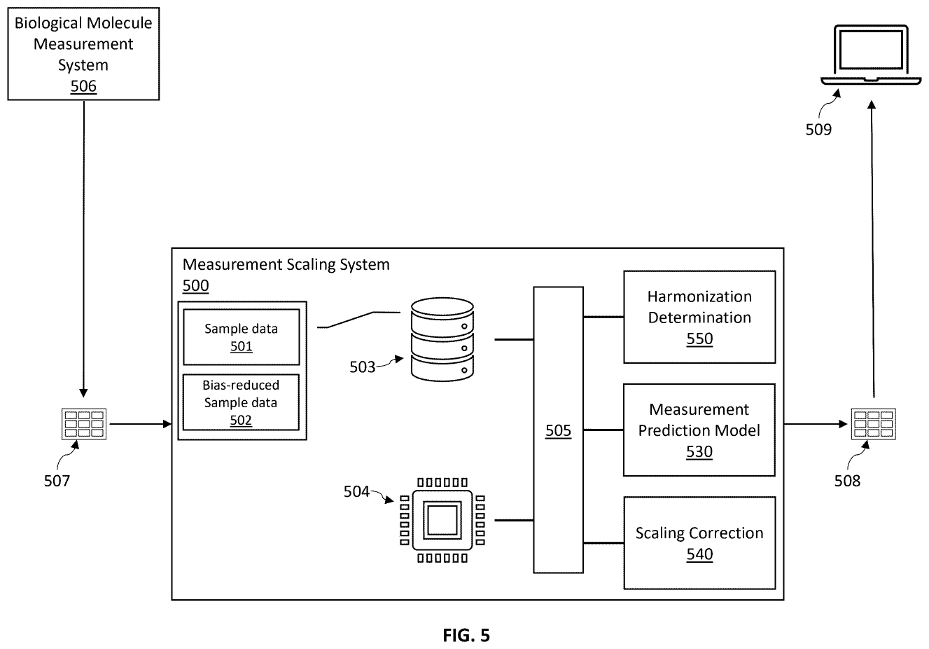

Referring now to FIG. 5, a block diagram depicts a system for scaling in biological molecule measurement systems in accordance with one or more embodiments of the present disclosure.

In some embodiments, a measurement scaling system 500 may curate, collect, obtain, access or otherwise use sample data 501 from biological measurements 507 collected by a biological molecule measurement system 506, such as any state biology including, e.g., a proteomics, genomics, metabolomics, antibody and/or peptide arrays, RNA sequencing or other measurement and/or analysis system. The sample data 501 may be stored in a database 503 and processed to produce bias-reduced sample data 502.

In some embodiments, the measurement scaling system 500 may include hardware components such as a processor(s) 504, which may include local or remote processing components. In some embodiments, the processor(s) 504 may include any type of data processing capacity, such as a hardware logic circuit, for example an application specific integrated circuit (ASIC) and a programmable logic, or such as a computing device, for example, a microcomputer or microcontroller that include a programmable microprocessor. In some embodiments, the processor(s) 504 may include data-processing capacity provided by the microprocessor. In some embodiments, the microprocessor may include memory, processing, interface resources, controllers, and counters. In some embodiments, the microprocessor may also include one or more programs stored in memory.

Similarly, the measurement scaling system 500 may include storage 503, such as one or more local and/or remote data storage solutions such as, e.g., local hard-drive, solid-state drive, flash drive, database or other local data storage solutions or any combination thereof, and/or remote data storage solutions such as a server, mainframe, database or cloud services, distributed database or other suitable data storage solutions or any combination thereof. In some embodiments, the storage 503 may include, e.g., a suitable non-transient computer readable medium such as, e.g., random access memory (RAM), read only memory (ROM), one or more buffers and/or caches, among other memory devices or any combination thereof.

In some embodiments, the measurement scaling system 500 may implement computer engines for harmonization determination 550, a measurement prediction model 530, and/or scaling correction 540. In some embodiments, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

In some embodiments, to determine anchoring molecules and/or measurements, the measurement scaling system 500 may include computer engines for the harmonization determination 550, such as for the harmonization determination 250/350 detailed above. In some embodiments, the computer engines for the harmonization determination 550 may include dedicated and/or shared software components, hardware components, or a combination thereof. For example, the computer engines for the harmonization determination 550 may include a dedicated processor and storage. However, in some embodiments, the computer engines for the harmonization determination 550 may share hardware resources, including the processor(s) 504 and storage 503 of the measurement scaling system 500 via, e.g., a bus 505.

In some embodiments, to determine expected or predicted measurements, the measurement scaling system 500 may include computer engines for the measurement prediction model 530, such as for the measurement prediction model 130, 230 and/or 430 detailed above. In some embodiments, the computer engines for the harmonization determination 550 may include dedicated and/or shared software components, hardware components, or a combination thereof. For example, the computer engines for the harmonization determination 550 may include a dedicated processor and storage. However, in some embodiments, the computer engines for the harmonization determination 550 may share hardware resources, including the processor(s) 504 and storage 503 of the measurement scaling system 500 via, e.g., a bus 505.

In some embodiments, to use expected or predicted measurements scaling (i.e., aligning), the measurement scaling system 500 may include computer engines for the scaling correction 540, such as for the scaling correction 140 detailed above. In some embodiments, the computer engines for the harmonization determination 550 may include dedicated and/or shared software components, hardware components, or a combination thereof. For example, the computer engines for the correlation determination 550 may include a dedicated processor and storage. However, in some embodiments, the computer engines for the harmonization determination 550 may share hardware resources, including the processor(s) 504 and storage 503 of the measurement bias reduction system 500 via, e.g., a bus 505.

In some embodiments, the measurement bias reduction system 500 may output a harmonized measurement dataset 508 for the biological measurements 507. The harmonized measurement dataset 508 may be output to a computing system 509 associated with a clinical or other user that is associated with the biological measurements 507.

In some embodiments, the user may be a clinician utilizing clinical decision support software of the computing system 509. In some embodiments, clinical decision support software (CDSS) may be a health information technology that provides clinicians, staff, patients, and other individuals with knowledge and person-specific information to help health and health care to enhance decision-making in the clinical workflow. The CDSS may include computerized alerts and reminders to care providers and patients, clinical guidelines, condition-specific order sets, focused patient data reports and summaries, documentation templates, diagnostic support, and contextually relevant reference information, among other tools.

In some embodiments, the CDSS of the computing system 509 may include a knowledge-based system, including, without limitation a knowledge base of protein measurements and/or proteomic data, an inference engine, and a mechanism to communicate. The knowledge base may include the rules and associations of protein measurements and/or proteomic data, including, without limitation, IF-THEN rules. The inference engine may combine the rules from the knowledge base with the harmonized measurement dataset 508 to produce clinical support recommendations, such as recommended diagnoses, triage recommendations, study design recommendations, among other clinical support tasks or any combination thereof.

In some embodiments, the CDSS may include non-knowledge-based CDSS which may not use a knowledge base, but may instead use machine learning or other artificial intelligence. Machine learning (ML) based CDSS may be used to generate similar clinical decision support, but since systems based on ML may not explain the reasons for their conclusions, the ML may instead be employed as post-diagnostic systems, for suggesting patterns for clinicians to look into in more depth.

For example, in some embodiments, a clinician, such as a doctor, may obtain medical data for one or more patients from a variety of different data sources. In one example, a doctor may obtain protein data for a patient at one time from one proteomics platform, and then protein data for the patient from another proteomics platform at a different time. Further, training of a machine learning model benefit from increased sizes of the training data set, which may be formed from multiple different measuring systems, platforms and/or devices. Thus, harmonization of data from different sources can be beneficial in practice, including via the use of an ML-based CDSS, such as predictive diagnostics, triage and risk classification systems or any combination thereof.

In some embodiments, the ML-based CDSS may include one or more AI and/or ML techniques, including those detailed further above, such as support-vector machines, artificial neural networks and genetic algorithms, among others or any combination thereof. For example, genetic algorithms, which are based on simplified evolutionary processes using directed selection to achieve optimal CDSS results, may use the selection algorithms to evaluate components of random sets of solutions to a problem based on the harmonized measurement dataset 508. The solutions that come out on top may then be recombined and mutated and run through the process again until the proper solution is discovered. In some embodiments, the ML-based CDSS may be trained on a static harmonized measurement dataset 508, or may be retrained on a live, or updating or growing harmonized measurement dataset 508 to refine the ML-based CDSS over time as more data is acquired.

In some embodiments, the computing system 509 may include software and/or systems for regulatory-compliant reporting. In some embodiments, the computing system 509 may update electronic health records (HER), insurance claims, drug study data, health study data, among other data records, with the harmonized measurement dataset 508. In some embodiments, the computing system 509 may include both the original biological measurements 507 as well as the harmonized measurement dataset 508 for auditability. As a result, the computing system 509 may be used to report data, including the harmonized measurement dataset 508, compliant with FDA, CLIA, or other regulatory standard or any combination thereof.

### Example—Harmonization of Proteomic Data

An example application of embodiments of data harmonization as detailed above can include combining cohorts of data, e.g., for use in medical study, predictive model training, among other uses or any combination thereof. An impediment to this application can include where a proteomics platform, such as, in this example, Olink™, may measure protein values in the first cohort that cannot be assumed to be numerically equivalent to those measured in a reference cohort (e.g., the UKB).

In some embodiments, as detailed above, harmonization can include out-of-band adjustment and in-band adjustment. Out-of-band adjustments use relationships between proteins and phenotypic or clinical lab values to align protein values between the reference COHORT and First cohorts. In-band adjustments use of untapped relationships among Olink-reported protein (NPX) values, to infer more accurate estimates. The focus of this is correcting errors introduced in the measurement process.

In some embodiments, out-of-band adjustments can include, in an example, body-mass index (BMI) from the protein Leptin. In the reference cohort, leptin can explain a significant fraction of the variance of BMI.

Referring to FIG. 6, predicted vs reported BMI in reference cohort are depicted in accordance with one or more embodiments of the present disclosure. As shown, the predicted BMI values, predicted using leptin as the out-of-band anchor, shows an R² value of 0.725.

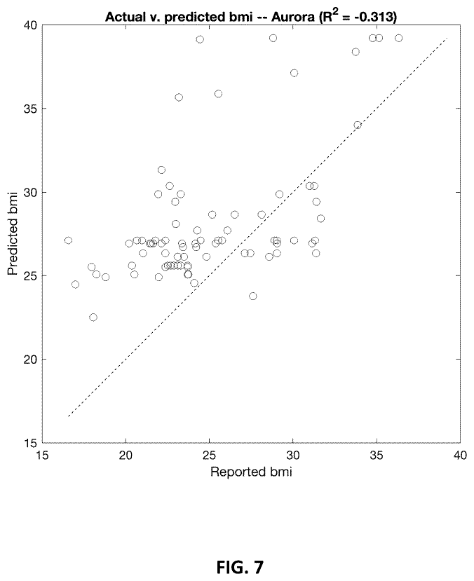

Referring to FIG. 7, predicted vs reported BMI in the first cohort are depicted in accordance with one or more embodiments of the present disclosure. In some embodiments, as shown, applying the same prediction model to the first cohort results in significantly worse performance.

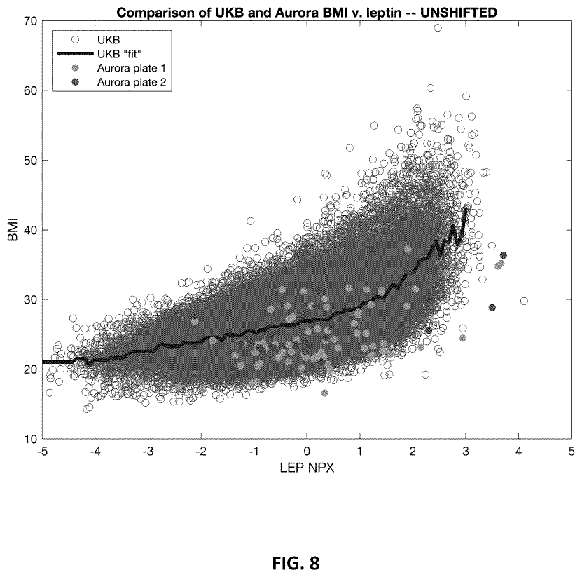

Referring to FIG. 8, BMI vs. leptin for reference cohort and first cohorts are depicted in accordance with one or more embodiments of the present disclosure. The plot of BMI vs leptin for both cohorts indicates that, relative to the reference cohort, the first cohort leptin values tend to be much higher for a given BMI. Thus, the first cohort predictions for BMI tend to be too high, which can be seen in FIG. 7.

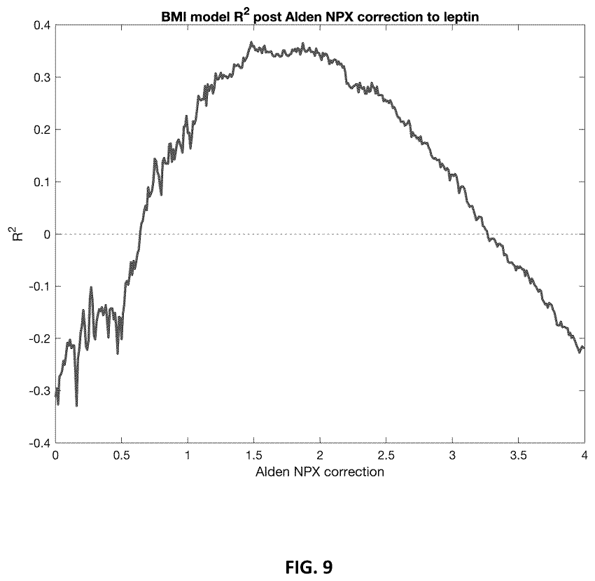

Referring to FIG. 9, model performance as a function of First cohort leptin NPX shift. Performance is optimized over a broad range of shift values, from around 1.5-2.1 are depicted in accordance with one or more embodiments of the present disclosure. Under the premise that it is the leptin values that are shifted in measurement relative to reference cohort measurements, the leptin values of the first cohort can be shifted to improve the prediction.

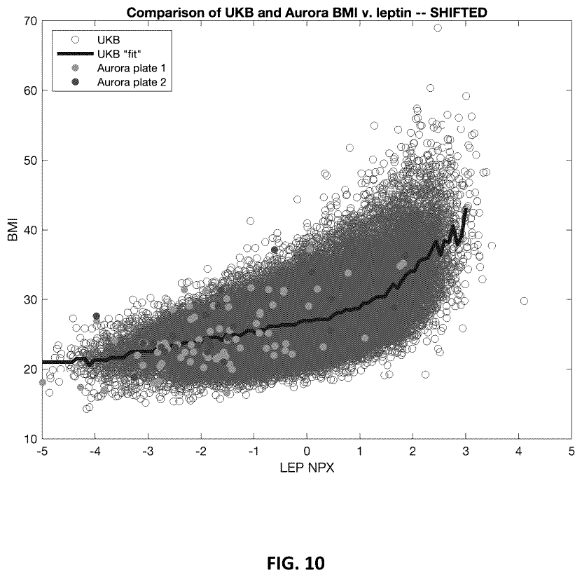

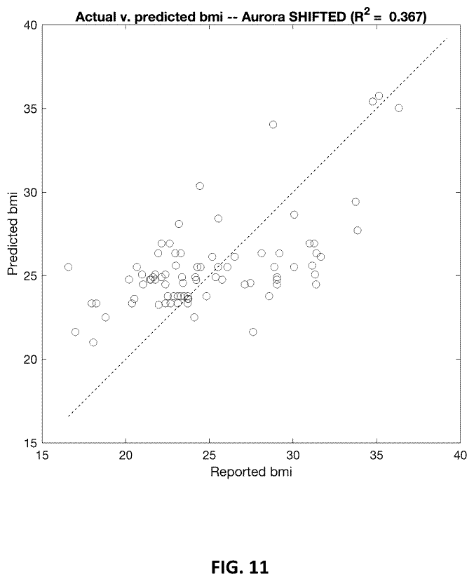

Referring to FIG. 10, BMI vs. leptin for reference COHORT and First cohorts, where the First cohort leptin value has been shifted (by 1.85 NPX units) to maximize model performance are depicted in accordance with one or more embodiments of the present disclosure. Relative to FIG. 7 above, shifting leptin by 1.85 (the center of the broad range of “optimal” shifts) brings the first cohort BMI vs leptin curve into alignment with the reference cohort. This shift improves model performance significantly (from R²=−0.31 to 0.37). While this performance is lower than the reference cohort performance, it does fall within the distribution of single-plate performance.

Referring to FIG. 11, predicted vs reported BMI in the first cohort, with shifted first cohort leptin values are depicted in accordance with one or more embodiments of the present disclosure. When compared to FIG. 7, the performance of the predictions after shifting the leptin values exhibits a performance improvement.

It is understood that at least one aspect/functionality of various embodiments described herein can be performed in real-time and/or dynamically. As used herein, the term “real-time” is directed to an event/action that can occur instantaneously or almost instantaneously in time when another event/action has occurred. For example, the “real-time processing,” “real-time computation,” and “real-time execution” all pertain to the performance of a computation during the actual time that the related physical process (e.g., a user interacting with an application on a mobile device) occurs, in order that results of the computation can be used in guiding the physical process.

As used herein, the term “dynamically” and term “automatically,” and their logical and/or linguistic relatives and/or derivatives, mean that certain events and/or actions can be triggered and/or occur without any human intervention. In some embodiments, events and/or actions in accordance with the present disclosure can be in real-time and/or based on a predetermined periodicity of at least one of: nanosecond, several nanoseconds, millisecond, several milliseconds, second, several seconds, minute, several minutes, hourly, several hours, daily, several days, weekly, monthly, etc.

As used herein, the term “runtime” corresponds to any behavior that is dynamically determined during an execution of a software application or at least a portion of software application.

In some embodiments, exemplary inventive, specially programmed computing systems and platforms with associated devices are configured to operate in the distributed network environment, communicating with one another over one or more suitable data communication networks (e.g., the Internet, satellite, etc.) and utilizing one or more suitable data communication protocols/modes such as, without limitation, IPX/SPX, X.25, AX.25, AppleTalk(TM), TCP/IP (e.g., HTTP), near-field wireless communication (NFC), RFID, Narrow Band Internet of Things (NBIOT), 3G, 4G, 5G, GSM, GPRS, WiFi, WiMax, CDMA, satellite, ZigBee, and other suitable communication modes.

In some embodiments, the NFC can represent a short-range wireless communications technology in which NFC-enabled devices are “swiped,” “bumped,” “tap” or otherwise moved in close proximity to communicate. In some embodiments, the NFC could include a set of short-range wireless technologies, typically requiring a distance of 10 cm or less. In some embodiments, the NFC may operate at 13.56 MHz on ISO/IEC 18000-3 air interface and at rates ranging from 106 kbit/s to 424 kbit/s. In some embodiments, the NFC can involve an initiator and a target; the initiator actively generates an RF field that can power a passive target. In some embodiments, this can enable NFC targets to take very simple form factors such as tags, stickers, key fobs, or cards that do not require batteries. In some embodiments, the NFC's peer-to-peer communication can be conducted when a plurality of NFC-enable devices (e.g., smartphones) within close proximity of each other.

The material disclosed herein may be implemented in software or firmware or a combination of them or as instructions stored on a machine-readable medium, which may be read and executed by one or more processors. A machine-readable medium may include any medium and/or mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Computer-related systems, computer systems, and systems, as used herein, include any combination of hardware and software. Examples of software may include software components, programs, applications, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Go, Perl, QT, etc.).

In some embodiments, one or more of illustrative computer-based systems or platforms of the present disclosure may include or be incorporated, partially or entirely into at least one personal computer (PC), laptop computer, ultra-laptop computer, tablet, touch pad, portable computer, handheld computer, palmtop computer, personal digital assistant (PDA), cellular telephone, combination cellular telephone/PDA, television, smart device (e.g., smart phone, smart tablet or smart television), mobile internet device (MID), messaging device, data communication device, and so forth.

As used herein, the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

In some embodiments, as detailed herein, one or more of the computer-based systems of the present disclosure may obtain, manipulate, transfer, store, transform, generate, and/or output any digital object and/or data unit (e.g., from inside and/or outside of a particular application) that can be in any suitable form such as, without limitation, a file, a contact, a task, an email, a message, a map, an entire application (e.g., a calculator), data points, and other suitable data. In some embodiments, as detailed herein, one or more of the computer-based systems of the present disclosure may be implemented across one or more of various computer platforms such as, but not limited to: (1) FreeBSD, NetBSD, OpenBSD; (2) Linux; (3) Microsoft Windows™; (4) Open VMS™; (5) OS X (MacOS™); (6) UNIX™; (7) Android; (8) iOS™; (9) Embedded Linux; (10) Tizen™; (11) WebOS™; (12) Adobe AIR™; (13) Binary Runtime Environment for Wireless (BREW™); (14) Cocoa™ (API); (15) Cocoa™ Touch; (16) Java™ Platforms; (17) JavaFX™. (18) QNX™; (19) Mono; (20) Google Blink; (21) Apple WebKit; (22) Mozilla Gecko™; (23) Mozilla XUL; (24) . NET Framework; (25) Silverlight™; (26) Open Web Platform; (27) Oracle Database; (28) Qt™; (29) SAP NetWeaver™; (30) Smartface™; (31) Vexi™; (32) Kubernetes™ and (33) Windows Runtime (WinRT™) or other suitable computer platforms or any combination thereof. In some embodiments, illustrative computer-based systems or platforms of the present disclosure may be configured to utilize hardwired circuitry that may be used in place of or in combination with software instructions to implement features consistent with principles of the disclosure. Thus, implementations consistent with principles of the disclosure are not limited to any specific combination of hardware circuitry and software. For example, various embodiments may be embodied in many different ways as a software component such as, without limitation, a stand-alone software package, a combination of software packages, or it may be a software package incorporated as a “tool” in a larger software product.

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

In some embodiments, illustrative computer-based systems or platforms of the present disclosure may be configured to handle numerous concurrent users that may be, but is not limited to, at least 100 (e.g., but not limited to, 100-999), at least 1,000 (e.g., but not limited to, 1,000-9,999), at least 10,000 (e.g., but not limited to, 10,000-99,999) , at least 100,000 (e.g., but not limited to, 100,000-999,999), at least 1,000,000 (e.g., but not limited to, 1,000,000-9,999,999), at least 10,000,000 (e.g., but not limited to, 10,000,000-99,999,999), at least 100,000,000 (e.g., but not limited to, 100,000,000-999,999,999), at least 1,000,000,000 (e.g., but not limited to, 1,000,000,000-999,999,999,999), and so on.

In some embodiments, illustrative computer-based systems or platforms of the present disclosure may be configured to output to distinct, specifically programmed graphical user interface implementations of the present disclosure (e.g., a desktop, a web app., etc.). In various implementations of the present disclosure, a final output may be displayed on a displaying screen which may be, without limitation, a screen of a computer, a screen of a mobile device, or the like. In various implementations, the display may be a holographic display. In various implementations, the display may be a transparent surface that may receive a visual projection. Such projections may convey various forms of information, images, or objects. For example, such projections may be a visual overlay for a mobile augmented reality (MAR) application.

In some embodiments, illustrative computer-based systems or platforms of the present disclosure may be configured to be utilized in various applications which may include, but not limited to, gaming, mobile-device games, video chats, video conferences, live video streaming, video streaming and/or augmented reality applications, mobile-device messenger applications, and others similarly suitable computer-device applications.

As used herein, the term “mobile electronic device,” or the like, may refer to any portable electronic device that may or may not be enabled with location tracking functionality (e.g., MAC address, Internet Protocol (IP) address, or the like). For example, a mobile electronic device can include, but is not limited to, a mobile phone, Personal Digital Assistant (PDA), Blackberry TM, Pager, Smartphone, or any other reasonable mobile electronic device.

As used herein, terms “proximity detection,” “locating,” “location data,” “location information,” and “location tracking” refer to any form of location tracking technology or locating method that can be used to provide a location of, for example, a particular computing device, system or platform of the present disclosure and any associated computing devices, based at least in part on one or more of the following techniques and devices, without limitation: accelerometer(s), gyroscope(s), Global Positioning Systems (GPS); GPS accessed using Bluetooth™; GPS accessed using any reasonable form of wireless and non-wireless communication; WiFi™ server location data; Bluetooth ™ based location data; triangulation such as, but not limited to, network based triangulation, WiFi™ server information based triangulation, Bluetooth™ server information based triangulation; Cell Identification based triangulation, Enhanced Cell Identification based triangulation, Uplink-Time difference of arrival (U-TDOA) based triangulation, Time of arrival (TOA) based triangulation, Angle of arrival (AOA) based triangulation; techniques and systems using a geographic coordinate system such as, but not limited to, longitudinal and latitudinal based, geodesic height based, Cartesian coordinates based; Radio Frequency Identification such as, but not limited to, Long range RFID, Short range RFID; using any form of RFID tag such as, but not limited to active RFID tags, passive RFID tags, battery assisted passive RFID tags; or any other reasonable way to determine location. For ease, at times the above variations are not listed or are only partially listed; this is in no way meant to be a limitation.

As used herein, terms “cloud,” “Internet cloud,” “cloud computing,” “cloud architecture,” and similar terms correspond to at least one of the following: (1) a large number of computers connected through a real-time communication network (e.g., Internet); (2) providing the ability to run a program or application on many connected computers (e.g., physical machines, virtual machines (VMs)) at the same time; (3) network-based services, which appear to be provided by real server hardware, and are in fact served up by virtual hardware (e.g., virtual servers), simulated by software running on one or more real machines (e.g., allowing to be moved around and scaled up (or down) on the fly without affecting the end user).

In some embodiments, the illustrative computer-based systems or platforms of the present disclosure may be configured to securely store and/or transmit data by utilizing one or more of encryption techniques (e.g., private/public key pair, Triple Data Encryption Standard (3DES), block cipher algorithms (e.g., IDEA, RC2, RC5, CAST and Skipjack), cryptographic hash algorithms (e.g., MD5, RIPEMD-160, RTRO, SHA-1, SHA-2, Tiger (TTH), WHIRLPOOL, RNGs).

As used herein, the term “user” shall have a meaning of at least one user. In some embodiments, the terms “user”, “subscriber” “consumer” or “customer” should be understood to refer to a user of an application or applications as described herein and/or a consumer of data supplied by a data provider. By way of example, and not limitation, the terms “user” or “subscriber” can refer to a person who receives data provided by the data or service provider over the Internet in a browser session, or can refer to an automated software application which receives the data and stores or processes the data.

Clause 1. A method comprising: obtaining, by at least one processor, first measurement of interest data from at least one first multi-omics data source, the first measurement of interest data comprising a first plurality of measurement of interest measurements for a first plurality of measurement of interests of a first plurality of subjects in a first data set; obtaining, by at least one processor, first out-of-band health-related measurement data comprising a first plurality of health-related measurements for the first plurality of subjects in the first data set; obtaining, by at least one processor, second measurement of interest data from at least one second multi-omics data source, the second measurement of interest data comprising a second plurality of measurement of interest measurements for a second plurality of measurement of interests of a second plurality of subjects in a second data set; obtaining, by at least one processor, second out-of-band health-related measurement data comprising a second plurality of health-related measurements for the second plurality of subjects in the second data set; identifying, by the at least one processor, one or more particular first subjects of the first plurality of subjects, and one or more particular second subjects of the second plurality of subjects, utilizing at least one machine learning model based on: the first plurality of health-related measurement measurements of the first out-of-band health-related measurement data, and the second plurality of health-related measurement measurements of the second out-of-band health-related measurement data; comparing, by the at least one processor, the first measurement of interest data with the second measurement of interest data based on the first plurality of measurement of interest measurements and the second plurality of measurement of interest measurements associated with the one or more particular second subjects; and harmonizing, by the at least one processor, the one or more particular second subjects with the plurality of subjects by adjusting the first plurality of measurement of interest measurements.

Clause 2. The method of clause 1, wherein the harmonizing comprises: dynamically scaling, by the at least one processor, the first plurality of measurement of interest measurements to match a standard deviation associated with the second plurality of measurement of interest measurements.

Clause 3. The method of clause 1, wherein the first out-of-band health-related measurement data and the second out-of-band health-related measurement data comprise genomic data.

Clause 4. The method of clause 1, wherein the at least one first multi-omics data platform comprises a proteomics platform.

Clause 5. The method of clause 1, further comprising applying, by the at least one processor, SNP-based effect size scaling to correct for batch effects and inter-platform variability in the first plurality of measurement of interest measurements.

Clause 6. The method of clause 1, wherein the first out-of-band health-related measurement data and the second out-of-band health-related measurement data comprise at least one of: genetic variants, polygenic risk scores, or genome-wide association study (GWAS).

The aforementioned examples are, of course, illustrative and not restrictive.

While one or more embodiments of the present disclosure have been described, it is understood that these embodiments are illustrative only, and not restrictive, and that many modifications may become apparent to those of ordinary skill in the art, including that various embodiments of the inventive methodologies, the illustrative systems and platforms, and the illustrative devices described herein can be utilized in any combination with each other. Further still, the various steps may be carried out in any desired order (and any desired steps may be added and/or any desired steps may be eliminated).

1. A method comprising:
obtaining, by at least one processor, first measurement of interest data from at least one first multi-omics data source, the first measurement of interest data comprising a first plurality of measurement of interest measurements for a first plurality of measurement of interests of a first plurality of subjects in a first data set;
obtaining, by at least one processor, first out-of-band health-related measurement data comprising a first plurality of anchor health-related measurements for the first plurality of subjects in the first data set;
obtaining, by at least one processor, second measurement of interest data from at least one second multi-omics data source, the second measurement of interest data comprising a second plurality of measurement of interest measurements for a second plurality of measurement of interests of a second plurality of subjects in a second data set;
obtaining, by at least one processor, second out-of-band health-related measurement data comprising a second plurality of anchor health-related measurements for the second plurality of subjects in the second data set;
identifying, by the at least one processor, one or more particular first subjects of the first plurality of subjects, and one or more particular second subjects of the second plurality of subjects, utilizing at least one machine learning model based at least in part on:
the first plurality of anchor health-related measurements of the first out-of-band health-related measurement data, and
the second plurality of anchor health-related a measurements of the second out-of-band health-related measurement data;
comparing, by the at least one processor, the first measurement of interest data with the second measurement of interest data based on the first plurality of measurement of interest measurements associated with the one or more particular first subjects and the second plurality of measurement of interest measurements associated with the one or more particular second subjects; and
harmonizing, by the at least one processor, the second data set with the first data set by:
determining based on the comparing, a scaling correction representing misalignment between the first measurement of interest data and the second measurement of interest data:
applying the scaling correction to the second plurality of measurement of interest measurements to generate a harmonized measurement of interest data set aligned with the first measurement of interest data, and
storing or outputting the harmonized measurement of interest data set as bias-reduced sample data.

2. The method of clause 1, wherein the harmonizing comprises: dynamically scaling, by the at least one processor, the second plurality of measurement of interest measurements to match a standard deviation associated with the first plurality of measurement of interest measurements.

3. The method of clause 1, wherein the first out-of-band health-related measurement data and the second out-of-band health-related measurement data comprise genomic data.

4. The method of clause 1, wherein the at least one first multi-omics data source comprises a proteomics platform.

5. The method of clause 1, further comprising applying, by the at least one processor, SNP-based effect size scaling to correct for batch effects and inter-platform variability in the first plurality of measurement of interest measurements.

6. The method of clause 1, wherein the first out-of-band health-related measurement data and the second out-of-band health-related measurement data comprise at least one of: genetic variants, polygenic risk scores, or genome-wide association study (GWAS).

7. The method of claim 1, wherein the identifying comprises selecting the one or more particular first subjects and the one or more particular second subjects based on a similarity measure between the first plurality of health-related measurements and the second plurality of health-related measurements.

8. The method of claim 1, wherein the machine learning model comprises a neural network trained on out-of-band health-related measurement data.

9. The method of claim 1, wherein the harmonizing further comprises adjusting the second plurality of measurement of interest measurements by applying a mean shift to align with the first plurality of measurement of interest measurements.

10. The method of claim 1, wherein the first out-of-band health-related measurement data and the second out-of-band health-related measurement data comprise at least one of: clinical laboratory measurements, phenotypic information, or metabolomic data.

11. A system for automated harmonization of biological sample measurements across multi-omics datasets, comprising:
at least one processor; and
memory storing instructions that, when executed by the at least one processor, cause the system to:
obtain first measurement-of-interest data from a first multi-omics data source and first out-of-band health-related measurement data for a first plurality of subjects in a first dataset;
obtain second measurement-of-interest data from a second multi-omics data source and second out-of-band health-related measurement data for a second plurality of subjects in a second dataset;
identify one or more particular first subjects of the first plurality of subjects and one or more particular second subjects of the second plurality of subjects via a machine-learned model based at least in part on the first and second out-of-band health-related measurement data;
for each of the one or more particular second subjects, select a set of neighbor subjects from the first plurality of subjects whose out-of-band health-related measurements exhibit similarity to corresponding out-of-band health-related measurements of the particular second subject;
determine reference measurement-of-interest statistics including at least a mean and a standard deviation for the selected neighbor subjects and subject measurement-of-interest statistics including at least a mean and a standard deviation for the particular second subject; and
adjust the second measurement-of-interest data by applying scale and shift operations to align the subject measurement-of-interest statistics with the reference measurement-of-interest statistics to generate a harmonized measurement of-interest data set, thereby harmonizing the second measurement-of-interest data with the first measurement-of-interest data, and store or output the harmonized measurement-of-interest data set as bias-reduced sample data.

12. The system of claim 11, wherein the out-of-band health-related measurements comprise genomic measurements including single nucleotide polymorphisms.

13. The system of claim 11, wherein the memory further stores instructions to pre-normalize the out-of-band health-related measurements by z-scoring within each dataset prior to neighbor selection.

14. The system of claim 11, wherein the similarity measure comprises a Euclidean distance computed between z-scored out-of-band health-related measurement vectors of respective subjects.

15. The system of claim 11, wherein the scale operation comprises multiplying the measurement-of-interest data by a ratio of the standard deviations of the reference measurement-of-interest statistics and the subject measurement-of-interest statistics.

16. The system of claim 11, wherein the shift operation comprises adding a difference between the means of the reference measurement-of-interest statistics and the subject measurement-of-interest statistics to the measurement-of-interest data.

17. The system of claim 11, wherein the memory further stores instructions that cause the system to evaluate harmonization performance by computing an R-squared value between predicted and reported health-related metric values.

18. The system of claim 11, wherein the out-of-band health-related measurements include one or more anchor measurements selected from body mass index, blood pressure, heart rate, clinical laboratory biomarkers, or polygenic risk scores.

19. The system of claim 11, wherein the harmonized measurement-of-interest data is output to a clinical decision support system for generating diagnostic or prognostic recommendations.

20. The system of claim 11, wherein the memory further stores instructions that cause the system to apply SNP-based effect size scaling to correct for batch effects and inter-platform variability in the measurement-of-interest data.
